(19) United States
(12) Patent Application Publication
(10) Pub. No.: US 2011/0053914 A1
(43) Pub. Date: Mar. 3, 2011

(54) Pharmaceutical Combination Comprising 6-Dimethylaminomethyl-1-(3-methoxy-phenyl)-cyclohexane-1,3-diol or 6-Dimethylaminomethyl-1-(3-hydroxy-phenyl)-cyclohexane-1,3-diol and an Antiepileptic

(75) Inventors: Klaus SCHIENE, Juechen (DE); Petra Bloms-Funke, Wuerselen (DE)

(73) Assignee: Gruenenthal GmbH, Aachen (DE)

(21) Appl. No.: 12/870,279

(22) Filed: Aug. 27, 2010

Related U.S. Application Data
(60) Provisional application No. 61/237,853, filed on Aug. 28, 2009.

(30) Foreign Application Priority Data
Aug. 28, 2009 (EP) 09 011 077.6

(51) Int. Cl. A61K 31/55; A61K 31/138; A61K 31/53; A61K 31/4015; A61K 31/165; A61K 31/27; A61P 29/00

(52) U.S. Cl. 514/217; 514/650; 514/242; 514/424; 514/616; 514/479

(57) ABSTRACT

A pharmaceutical combination comprising:

(a) at least one 6-dimethylaminomethyl-1-(3-methoxy-phenyl)-cyclohexane-1,3-diol component and/or at least one 6-dimethylaminomethyl-1-(3-hydroxy-phenyl)-cyclohexane-1,3-diol component, and

(b) at least one antiepileptic; a pharmaceutical composition comprising said combination,

a medicament comprising such a combination, a kit containing the ingredients of such a combination, and a method of treating pain in which components (a) and (b) are administered simultaneously or sequentially, whereby component (a) may be administered before or after component (b), and in which components (a) and (b) are administered either via the same or a different pathway of administration.

### CROSS REFERENCE TO RELATED APPLICATIONS

The present application claims priority from co-pending U.S. provisional patent application No. 61/237,853, filed Aug. 28, 2009. Priority is also claimed based on European patent application no. EP 09 011 077.6, also filed Aug. 28, 2009.

### BACKGROUND OF THE INVENTION

The present invention relates to a combination comprising (a) at least one 6-dimethylaminomethyl-1-(3-methoxy-phenyl)-cyclohexane-1,3-diol component and/or at least one 6-dimethylaminomethyl-1-(3-hydroxy-phenyl)cyclohexane-1,3-diol component, and (b) at least one antiepileptic; a pharmaceutical composition comprising said combination, a medicament comprising said combination, a kit as well as a method of treating pain wherein components (a) and (b) are administered simultaneously or sequentially, wherein component (a) may be administered before or after component (b) and wherein components (a) or (b) are administered either via the same or a different pathway of administration.

The treatment of pain conditions is extremely important in medicine. There is currently a worldwide demand for additional, not exclusively opioid-based, but highly effective pain treatment. The urgent need for action for patient-oriented and purposeful treatment of pain conditions, this being taken to mean the successful and satisfactory treatment of pain for the patient, is documented in the large number of scientific papers which have recently appeared in the field of applied analgesics and fundamental research work on nociception.

Even if the analgesics that are currently used for treating pain, for example opioids, NA- and 5HT-reuptake inhibitors, NSAIDS and COX inhibitors, are analgesically effective, side effects nevertheless sometimes occur. WO 01/13904 describes substance combinations comprising a tramadol material and an anticonvulsant drug, which show super-additive effects upon administration. Due to the super-additive effect the overall dose and accordingly the risk of undesired side effects can be reduced.

### SUMMARY OF THE INVENTION

Thus, it was an object of the present invention to find further combinations that are suitable for the treatment of pain and which preferably exhibit fewer undesired side effects compared to its individual components, if administered in effective doses.

It has been found that a combination comprising (a) at least one 6-dimethylaminomethyl-1-(3-methoxy-phenyl)cyclohexane-1,3-diol component, and (b) at least one antiepileptic exhibits an analgesic effect. Thus, said combination is particularly useful for the treatment of pain. If the components are present in the composition in such a weight ratio that a synergistic effect is observed upon administration to the patient, the overall administered dose may be lowered, so that fewer undesired side-effects will occur.

Accordingly, in one of its embodiments the present invention relates to a combination comprising:

(a) at least one 6-dimethylaminomethyl-1-(3-methoxy-phenyl)-cyclohexane-1,3-diol of formula (I)

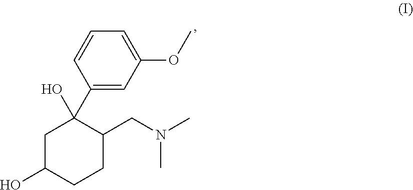

or a salt thereof, and/or at least one 6-dimethylaminomethyl-1-(3-hydroxy-phenyl)-cyclohexane-1,3-diol of formula (I′)

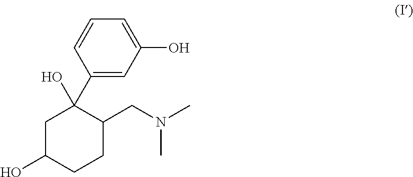

or a salt thereof, and

(b) at least one antiepileptic.

The compound of formula (I) or a metabolite thereof, i.e. a compound of formula (I′) may be present in form of one of its stereoisomers, in particular an enantiomer or a diastereomer, a racemate or in form of a mixture of its stereoisomers, in particular enantiomers and/or diastereomers in any mixing ratio, or any corresponding salt or solvate.

In yet another embodiment the inventive combination comprises

(a) (1RS,3RS,6RS)-6-dimethylaminomethyl-1-(3-methoxy-phenyl)-cyclohexane-1,3-diol, i.e. a mixture of the compounds (Ia and Ib):

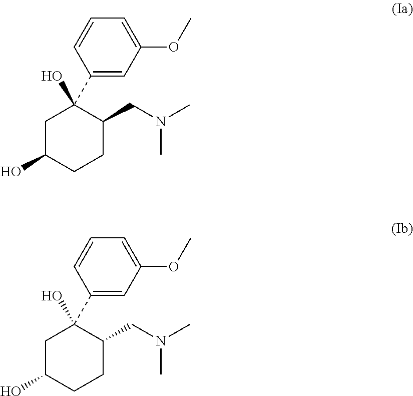

or a salt thereof, and/or (1RS,3RS,6RS)-6-dimethylaminomethyl-1-(3-hydroxy-phenyl)-cyclohexane-1,3-diol, i.e. a mixture of the compounds (IIa and IIb)

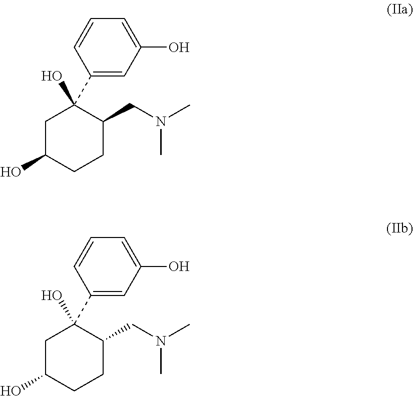

or a salt thereof, and

(b) at least one antiepileptic.

As used herein (1RS,3RS,6RS)-6-dimethylaminomethyl-1-(3-methoxy-phenyl)-cyclohexane-1,3-diol designates a mixture of the compounds of formulas Ia and Ib, wherein said mixture may preferably be racemic.

As used herein (1RS,3RS,6RS)-6-dimethylaminomethyl-1-(3-hydroxy-phenyl)-cyclohexane-1,3-diol designates a mixture of the compounds of formulas IIa and IIb, wherein said mixture may preferably be racemic.

In one of its preferred embodiments the present invention relates to a combination comprising

(a) at least one 6-dimethylaminomethyl-1-(3-methoxy-phenyl)-cyclohexane-1,3-diol of formula (I)

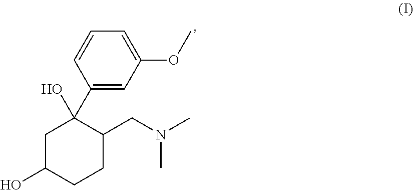

or a salt thereof, and

(b) at least one antiepileptic.

In yet another preferred embodiment the inventive combination comprises

(a) a mixture of compounds (Ia) and (Ib),

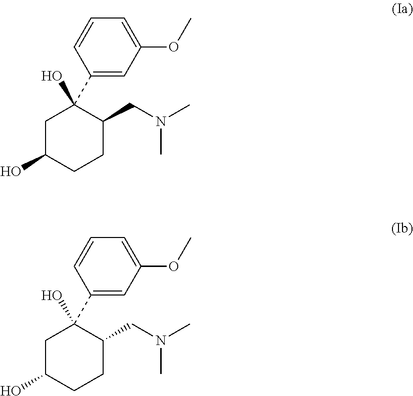

or a salt thereof, and

(b) at least one antiepileptic.

In the afore mentioned combination the mixture of compounds (Ia) and (Ib) may preferably be racemic.

The 6-dimethylaminomethyl-1-(3-methoxy-phenyl)-cyclohexane-1,3-diol of formula (I), its stereoisomers such as (1RS,3RS,6RS)-6-dimethylaminomethyl-1-(3-methoxy-phenyl)-cyclohexane-1,3-diol, and corresponding salts thereof as well as methods for their preparation are well known, for example, from U.S. RE 37,355 E. The respective parts of the description are hereby incorporated by reference and form part of the present disclosure.

The specific racemic compound (1RS,3RS,6RS)-6-dimethylaminomethyl-1-(3-methoxy-phenyl)-cyclohexane-1,3-diol is also known as axomadol (WHO Drug Information, Vol. 16, No. 2, 2002, List 87), which is a synthetic, centrally active analgesic that is suitable for the treatment of moderate to severe, acute or chronic pain. Axomadol can be used in the form of its free base or as a salt or solvate.

If any of the components, particularly component (a), is present as mixture of enantiomers, such a mixture may contain the enantiomers in racemic or non-racemic form. A non-racemic form could, for example, could contain the enantiomers in a ratio of 60±5:40±5; 70±5:30±5; 80±5:20±5 or 90±5:10±5.

The 6-dimethylaminomethyl-1-(3-methoxy-phenyl)-cyclohexane-1,3-diol, 6-dimethylaminomethyl-1-(3-hydroxy-phenyl)-cyclohexane-1,3-diol and their respective stereoisomers, in particular (1RS,3RS,6RS)-6-dimethylaminomethyl-1-(3-methoxy-phenyl)cyclohexane-1,3-diol, according to component (a) may be present in the inventive combination in form of a salt, preferably an acid addition salt, whereby any suitable acid capable of forming such a salt may be used.

Suitable pharmaceutically compatible salts of these compounds, particularly of (1RS,3RS,6RS)-6-dimethylaminomethyl-1-(3-methoxy-phenyl)-cyclohexane-1,3-diol, include salts of inorganic and/or organic acids such as e.g. acetic acid, 2,2-dichloroacetic acid, acylated amino acids, preferably acetylated amino acids such as e.g. N-acetylalanine, N-acetylcysteine, N-acetylglycine, N-acetylisoleucine, N-acetylleucine, N-acetylmethionine, N-acetylphenylalanine, N-acetylproline, N-acetylserine, N-acetylthreonine, N-acetyltyrosine, N-acetylvaline, adipic acid, alginic acid, ascorbic acid, L-aspartic acid, benzenesulfonic acid, benzoic acid, 4-acetamidobenzoic acid, (+)-camphoric acid, (−)-camphoric acid, (+)-camphor sulfonic acid, (−)-camphor sulfonic acid, (+)-camphor-10-sulfonic acid, (−)-camphor-10-sulfonic acid, (±)-camphor-10-sulfonic acid, capric acid, caproic acid, caprylic acid, cation exchange resins, cinnamic acid, citric acid, cyclohexyl sulfamic acid, sulfuric acid monododecyl ester, ethane-1,2-sulfonic acid, ethanesulfonic acid, 2-hydroxyethanesulfonic acid, formic acid, fumaric acid, mucic acid (galactosaccharic acid), gentisic acid, glucose monocarboxylic acid (glucoheptonic acid), d-gluconic acid, d-glucuronic acid, L-glutamic acid, α-oxoglutaric acid (α-ketoglutaric acid), hydroxyacetic acid (glycollic acid), hippuric acid (N-benzoylglycine), hydrogen bromide, hydrogen chloride, (+)-L-lactic acid, (±)-DL-lactic acid, lactobionic acid (4-O-β-D-galactopyranosyl-D-gluconic acid), maleic acid, (−)-L-malic acid, malonic acid, (±)-DL-mandelic acid, methanesulfonic acid, naphthalene-2-sulfonic acid, naphthalene-2,5-disulfonic acid, 1-hydroxy-2-naphthalene-carboxylic acid, nicotinic acid, nitric acid, oleic acid, orotic acid (uracil-6-carboxylic acid), oxalic acid, palmitic acid, pamoa acid (embonic acid), phosphoric acid, L-pyroglutamic acid, salicylic acid, acetylsalicylic acid, 4-aminosalicylic acid, sebacic acid, stearic acid, succinic acid, sulfuric acid, tannic acid, (+)-L-tartaric acid, (±)-DL-tartaric acid, thiocyanic acid, p-toluenesulfonic acid and undecylenic acid. Preferred salts are hydrochloride, saccharinate, dihydrogen phosphate, hydrogen phosphate and phosphate.

The aforementioned compounds, in particular (1RS,3RS,6RS)-6-dimethylaminomethyl-1-(3-methoxy-phenyl)-cyclohexane-1,3-diol, can also be present as a mixture of salts of the above-mentioned organic and inorganic acids in any desired ratio.

Antiepileptics, which are often also referred to as anticonvulsants, are well known in the art and include, without limitation, barbiturates and derivatives, such as methylphenobarbital, phenobarbital, primidone, barbexaclone and metharbital; hydantoin derivatives such as ethotoin, phenyloin, amino(diphenylhydantoin) valeric acid, mephenyloin and fosphenyloin; oxazolidine derivatives such as paramethadione, trimethadione and ethadione; succinimide derivatives such as ethosuximide, phensuximide and mesuximide; benzodiazepine derivatives such as clonazepam; carboxamide derivatives such as carbamazepine, oxcarbazepine, eslicarbazepine and rufinamide; fatty acid derivatives such as valproic acid, valpromide, aminobutyric acid, vigabatrin, progabide and tiagabine; or other antiepileptics such as sultiame, phenacemide, lamotrigine, felbamate, topiramate, gabapentin, pheneturide, levetiracetam, brivaracetam, seletracetam, zonisamide, pregabaline, stiripentol, lacosamide and beclamide.

These aforementioned classes of antiepileptics and most of their individual representatives are, for example, listed in the Anatomical Therapeutic Chemical (ATC) classification under [N03] as used by the WHO for classification of pharmaceutically active ingredients (preferred edition: January 2008 or 2009). With regard to further details of the ATC-index reference is made to U. Fricke, J. Günther, Anatomisch-therapeutisch-chemische Klassifikation mit Tagesdosen für den deutschen Arzneimittelmarkt: Methodik der ATC-Klassifikation and DDD-Festlegung. ATC-Index mit DDD-Angaben, Wissenschaftliches Institut der AOK; and Swiss Pharmaceutical Society, Index Nominum: International Drug Directory, CRC Press; 18th edition (Jan. 31, 2004).

Other suitable antiepileptics include, for example, retigabine, carisbamate, perampanel and ralfinamide.

Some antiepileptics are known to be useful in the treatment of neuropathic pain. In one embodiment of the present invention one or more of these antiepileptics is used as component (b).

Also included are stereoisomers, salts, solvates, and derivatives of the antiepileptic component as well as mixtures of any of the foregoing.

In one embodiment of the inventive combination the antiepileptic according to component (b) is selected from the group consisting of pregabaline, gabapentin, topiramate, lamotrigine, carbamazepine, oxcarbamazepine, eslicarbazepine, lacosamide, phenyloin, levetiracetam, brivaracetam, seletracetam, retigabine, carisbamate, perampanel, valproic acid and ralfinamide.

In another embodiment of the inventive combination the antiepileptic according to component (b) is selected from the group consisting of pregabaline, gabapentin, topiramate, lamotrigine, carbamazepine, lacosamide, phenyloin, levetiracetam, retigabine, carisbamate, perampanel, valproic acid and ralfinamide.

In another embodiment of the inventive combination the antiepileptic according to component (b) is pregabaline.

In yet another embodiment of the inventive combination the antiepileptic according to component (b) is (S)-pregabaline.

A specific embodiment of the present invention is a combination comprising (a) (1RS,3RS,6RS)-6-dimethylaminomethyl-1-(3-methoxy-phenyl)-cyclohexane-1,3-diol, or a salt thereof such as the hydrochloride, saccharinate, dihydrogen phosphate, hydrogen phosphate or phosphate, and (b) one or more antiepileptics selected from the group consisting of pregabaline, gabapentin, topiramate, lamotrigine, carbamazepine, oxcarbamazepine, eslicarbazepine, lacosamide, phenyloin, levetiracetam, brivaracetam, seletracetam, retigabine, carisbamate, perampanel, valproic acid and ralfinamide.

Another specific embodiment of the present invention is a combination comprising (a) (1RS,3RS,6RS)-6-dimethylaminomethyl-1-(3-methoxy-phenyl)-cyclohexane-1,3-diol, or a salt thereof such as the hydrochloride, saccharinate, dihydrogen phosphate, hydrogen phosphate or phosphate, and (b) one or more antiepileptics selected from the group consisting of pregabaline, gabapentin, topiramate, lamotrigine, carbamazepine, lacosamide, phenyloin, levetiracetam, retigabine, carisbamate, perampanel, valproic acid and ralfinamide.

Yet another specific embodiment of the present invention is a combination comprising (a) (1RS,3RS,6RS)-6-dimethylaminomethyl-1-(3-methoxy-phenyl)-cyclohexane-1,3-diol, or a salt thereof such as the hydrochloride, saccharinate, dihydrogen phosphate, hydrogen phosphate or phosphate, and (b) pregabaline.

A further specific embodiment of the present invention is a combination comprising (a) (1RS,3RS,6RS)-6-dimethylaminomethyl-1-(3-methoxy-phenyl)-cyclohexane-1,3-diol, or a salt thereof such as the hydrochloride, saccharinate, dihydrogen phosphate, hydrogen phosphate or phosphate, and (b) (S)-pregabaline.

Still yet another specific embodiment of the present invention is a combination comprising (a) (1RS,3RS,6RS)-6-dimethylaminomethyl-1-(3-methoxy-phenyl)-cyclohexane-1,3-diol, or the hydrochloride salt thereof, and (b) pregabaline.

Another specific embodiment of the present invention is a combination comprising (a) (1RS,3RS,6RS)-6-dimethylaminomethyl-1-(3-methoxy-phenyl)-cyclohexane-1,3-diol, or its hydrochloride salt thereof, and (b) (S)-pregabaline.

Some antiepileptics comprise functional groups, for example, acidic groups such as carboxy groups which are capable of forming salts or covalent compounds with the 6-dimethylaminomethyl-1-(3-methoxy-phenyl)-cyclohexane-1,3-diol, in particular with (1RS,3RS,6RS)-6-dimethylaminomethyl-1-(3-methoxy-phenyl)-cyclohexane-1,3-diol, thereby incorporating both components (a) and (b) in one and the same salt or compound.

Thus, in another embodiment of the present invention the inventive combination comprises components (a) and (b) in form of a salt or a preferably covalent compound formed from these two components. Such a salt or compound formation may be partial, i.e. the inventive composition comprises one or both of these components also in their free form, or the formation may essentially be complete.

The quantities of components (a) and (b) to be administered to patients vary depending on the weight of the patient, the type of application and the severity of the illness. Both components (a) and (b) as part of the inventive combination may be administered in amounts up to their maximum daily dosage, which is known to those skilled in the art.

Preferably the 6-dimethylaminomethyl-1-(3-methoxy-phenyl)cyclohexane-1,3-diol compound, in particular (1RS,3RS,6RS)-6-dimethylaminomethyl-1-(3-methoxy-phenyl)-cyclohexane-1,3-diol, in each case based on the free base, may be administered to a patient in a daily dosage of 10 to 2000 mg, particularly preferably in a dosage of 15 to 1500 mg, more particularly preferably in a dosage of 20 to 1000 mg.

The compound pregabaline may preferably be administered to a patient in a daily dosage of 1 to 1200 mg, the compound Gabapentin may preferably be administered to a patient in a daily dosage of 1 to 5000 mg, in each case based on the free base.

When administered as part of the inventive combination the administered amount per day of component (a) and/or component (b) may be less than the respective maximum daily dosage and be, for example, 75±15 wt.-%, 75±10 wt.-%, 75±5 wt.-%, 50±15 wt.-%, 50±10 wt.-%, 50±5 wt.-%, 25±15 wt.-%, 25±10 wt.-% and 25±5 wt.-% for each of the components.

In another embodiment of the present invention the inventive combination may contain components (a) and (b) essentially in an equieffective ratio.

In yet a further embodiment of the inventive combination components (a) and (b) are present in such a weight ratio that the resulting composition will exert a synergistic effect upon administration to a patient. Suitable weight ratios can be determined by methods well known to those skilled in the art.

Both components (a) and (b) may also be present in the inventive combination in ratios deviating from the equieffective ratio. For, example, each of the components could be present in a range from 1/50 of the equieffective amount to 50 times the equieffective amount, from 1/20 of the equieffective amount to 20 times the equieffective amount, from 1/10 of the equieffective amount to 10 times the equieffective amount, from ⅕ of the equieffective amount to 5 times the equieffective amount, from ¼ of the equieffective amount to 4 times the equieffective amount, from ⅓ of the equieffective amount to 3 times the equieffective amount, or from ½ of the equieffective amount to 2 times the equieffective amount.

In one of its embodiments the present invention relates to an inventive combination of components (a) and (b) as described herein for the treatment of pain.

The term pain as used herein preferably includes but is not limited to pain selected from the group consisting of inflammatory pain, pain associated with arthrosis, postoperative pain, neuropathic pain, acute pain, chronic pain, visceral pain, headache, migraine pain and cancer pain.

Different types of pain associated with arthrosis are described, for example, in WO 2008/138558 the respective contents of which hereby being incorporated by reference and forming part of the disclosure of the present invention.

In another embodiment the present invention relates to a pharmaceutical composition comprising at least one of the inventive combinations of components (a) and (b) described herein and optionally one or more suitable additives and/or adjuvants such as described below. Preferably said pharmaceutical composition may be used for the treatment of pain.

In another embodiment the present invention relates to a medicament comprising at least one of the inventive combinations of components (a) and (b) described herein. In a preferred embodiment, the medicament is a solid drug form. The medicament is preferably manufactured for oral administration. However, other forms of administration are also possible, e.g. for buccal, sublingual, transmucosal, rectal, intralumbal, intraperitoneal, transdermal, intravenous, intramuscular, intragluteal, intracutaneous and subcutaneous application.

Depending on the configuration, the medicament (dosage form) preferably contains suitable additives and/or adjuvants. Suitable additives and/or adjuvants in the sense of the invention are all substances known to a person skilled in the art for the formation of galenic formulations. The choice of these adjuvants and also the quantities to be used are dependent on how the medication is to be administered, i.e. orally, intravenously, intraperitoneally, intradermally, intramuscularly, intranasally, buccally or locally.

Preparations suitable for oral administration are those in the form of tablets, chewable tablets, lozenges, capsules, granules, drops, liquids or syrups, and those suitable for parenteral, topical and inhalatory administration are solutions, suspensions, easily reconstituted dry preparations and sprays. A further possibility is suppositories for rectal administration. The application in a depot in dissolved form, a patch or a plaster, possibly with the addition of agents promoting skin penetration, are examples of suitable percutaneous forms of administration.

Examples of adjuvants and additives for oral forms of application include disintegrants, lubricants, binders, fillers, mould release agents, possibly solvents, flavourings, sugar, in particular carriers, diluents, colouring agents, antioxidants etc.

Waxes or fatty acid esters, amongst others, can be used for suppositories and carrier substances, preservatives, suspension aids etc. can be used for parenteral forms of application.

Suitable adjuvants include, for example, water, ethanol, 2-propanol, glycerine, ethylene glycol, propylene glycol, polyethylene glycol, polypropylene glycol, glucose, fructose, lactose, saccharose, dextrose, molasses, starch, modified starch, gelatine, sorbitol, inositol, mannitol, microcrystalline cellulose, methyl cellulose, carboxymethyl-cellulose, cellulose acetate, shellac, cetyl alcohol, polyvinylpyrrolidone, paraffins, waxes, natural and synthetic rubbers, acacia gum, alginates, dextran, saturated and unsaturated fatty acids, stearic acid, magnesium stearate, zinc stearate, glyceryl stearate, sodium lauryl sulfate, edible oils, sesame oil, coconut oil, peanut oil, soybean oil, lecithin, sodium lactate, polyoxyethylene and propylene fatty acid esters, sorbitane fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talc, kaolin, pectin, crospovidon, agar and bentonite.

The production of these medicaments and pharmaceutical compositions is conducted using means, devices, methods and processes that are well known in the art of pharmaceutical technology, as described, for example, in “Remington's Pharmaceutical Sciences”, A. R. Gennaro, 17th ed., Mack Publishing Company, Easton, Pa. (1985), in particular in part 8, chapters 76 to 93.

Thus, for example, for a solid formulation such as a tablet, the active substances of the drug can be granulated with a pharmaceutical carrier substance, e.g. conventional tablet constituents such as cornstarch, lactose, saccharose, sorbitol, talc, magnesium stearate, dicalcium phosphate or pharmaceutically acceptable rubbers, and pharmaceutical diluents such as water, for example, in order to form a solid composition that contains the active substance in a homogenous dispersion. Homogenous dispersion is understood here to mean that the active substances are uniformly dispersed throughout the composition, so that this can be readily divided into identically effective standard dosage forms such as tablets, capsules, lozenges. The solid composition is then divided into standard dosage forms. The tablets or pills can also be coated or otherwise compounded to prepare a slow release dosage form. Suitable coating agents include polymeric acids and mixtures of polymeric acids with materials such as shellac, cetyl alcohol and/or cellulose acetate, for example.

In another preferred embodiment of the present invention one or both of the components (a) and (b) is/are present in immediate release form.

In another preferred embodiment of the present invention one or both of the components (a) and (b) is/are present in controlled-release form. In particular, components (a) and (b) can be released slowly from preparations that can be applied orally, rectally or percutaneously.

The medicament can preferably be manufactured for administration once daily, twice daily (bid), or three times daily, the once daily or twice daily administration (bid) being preferred.

The term controlled release as used herein refers to any type of release other than immediate release such as delayed release, sustained release, slow release, extended release and the like. These terms are well known to any person skilled in the art as are the means, devices, methods and processes for obtaining such type of release.

A controlled release of any of the components (a) and (b) can be achieved, for example, by retardation using a matrix, a coating or osmotically active release systems such as described for axomadol in WO 2005/009329, for example. The respective parts of the description are hereby incorporated by reference and form part of the present disclosure.

If one of the components is to be released prior to the other component, for example at least 15 minutes, 30 minutes, 45 minutes or 1 hour beforehand, medicaments having a corresponding release profile may be prepared. An example of such a formulation is an osmotically driven release system for achieving a delayed release of one component via a coating that itself contains the other component which is accordingly released earlier. In a release system of this kind, which is particularly suitable for oral administration, at least part, and preferably all, of the surface of the release system, preferably those parts that will come into contact with the release medium, is/are semipermeable, preferably equipped with a semipermeable coating, so the surface(s) is/are permeable to the release medium, but substantially, preferably entirely, impermeable to the active ingredient, the surface(s) and/or optionally the coating comprising at least one opening for releasing the active ingredient. Moreover, precisely that/those surface(s) that is/are in contact with the release medium is/are provided with a coating containing and releasing the other component. This is preferably taken to mean a system in tablet form comprising a release opening, an osmotic pharmaceutical composition core, a semipermeable membrane and a polymer portion that exerts pressure upon swelling. A suitable example of this kind of system is the system distributed by ALZA Corporation, USA under the tradenames OROS®, in particular, the OROS® Push-Pull™ System, the OROS® Delayed Push-Pull™ System, the OROS® Multi-Layer Push-Pull™ system, the OROS® Push-Stick System and also, in specific cases, the L-OROS™.

Embodiments and examples of osmotically driven release systems are, for example, disclosed in U.S. Pat. Nos. 4,765,989, 4,783,337 and 4,612,008, all of the respective contents of which are hereby incorporated by reference and form part of the disclosure of the present invention.

A further example of a suitable pharmaceutical formulation is a gel-matrix tablet, such as the products developed by Penwest Pharmaceuticals (for example, under TimeRX). Suitable examples are provided in U.S. Pat. Nos. 5,330,761, 5,399,362, 5,472,711 and 5,455,046, all of the respective contents of which are hereby incorporated by reference and form part of the disclosure of the present application. Particularly suitable is a retarding matrix formulation, with an inhomogeneous distribution of the pharmaceutically active composition, whereby, for example, one component can be distributed in the outer region (the portion that comes into contact with the release medium most quickly) of the matrix and the other component is distributed inside the matrix. On contact with the release medium, the outer matrix layer initially (and rapidly) swells and initially releases the first component, followed by the significantly (more) retarded release of the other component. Examples of a suitable matrix include matrices with 1 to 80% by weight of one or more hydrophilic or hydrophobic polymers as pharmaceutically acceptable matrix formers. A further example of a suitable matrix may be inferred from U.S. Pat. No. 4,389,393, the respective contents of which are hereby incorporated by reference and form part of the disclosure of the present application.

In another preferred embodiment of the present invention
the medicament is manufactured for oral administration; and/or
the medicament is a solid and/or compressed and/or film-coated drug form; and/or
the medicament releases one or both components (a) and (b) slowly from a matrix; and/or
the medicament contains both components (a) and (b) in a quantity of 0.001 to 99.999% by wt., more preferred 0.1 to 99.9% by wt., still more preferred 1.0 to 99.0% by wt., even more preferred 2.5 to 80% by wt., most preferred 5.0 to 50% by wt. and in particular 7.5 to 40% by wt., based on the total weight of the medicament; and/or
the medicament contains a pharmaceutically compatible carrier and/or pharmaceutically compatible adjuvants; and/or
the medicament has a total mass in the range of 25 to 2000 mg, more preferred 50 to 1800 mg, still more preferred 60 to 1600 mg, more preferred 70 to 1400 mg, most preferred 80 to 1200 mg and in particular 100 to 1000 mg; and/or
the medicament is selected from the group comprising tablets, capsules, pellets and granules.

The medicament can be provided as a simple tablet and as a coated tablet (e.g. as film-coated tablet or lozenge). The tablets are usually round and biconvex, but oblong forms are also possible. Granules, spheres, pellets or microcapsules, which are contained in sachets or capsules or are compressed to form disintegrating tablets, are also possible.

In yet another one of its embodiments, the present invention relates to the use of at least one of the inventive combinations of components (a) and (b) described herein for the production of a medicament. Preferably said medicament is suitable for the treatment of pain.

In still another one of its embodiment, the present invention relates to the use of at least one of the inventive combinations of components (a) and (b) described herein for the treatment of pain.

Furthermore, the present invention relates to a method for treating pain in a patient, preferably in a mammal, which comprises administering an effective amount of at least one inventive combination as described herein to a patient.

According to said method components (a) and (b) are administered simultaneously or sequentially to a patient, preferably a mammal, wherein component (a) may be administered before or after component (b) and wherein components (a) or (b) are administered to the patient, preferably a mammal, either via the same or a different pathway of administration.

The present invention also relates to a kit comprising one or more of the above-described medicaments (dosage forms) according to the invention.

The kit according to the invention is preferably designed for in each case once daily, twice daily or three times daily administration of the medicaments (dosage forms) contained therein.

In one embodiment the kit according to the invention comprises one or more medicaments configured for administering components (a) and (b) simultaneously or sequentially, whereby compound (a) may be administered before or after compound (b) and wherein components (a) or (b) may be administered by the same or a different pathway of administration.

The following examples serve to explain the invention in more detail, but should not be interpreted as restrictive.

### Pharmacological Methods:

### 1. Paw Incision Model in Rats

The paw incision model (Brennan, T. J., Vandermeulen, E. P., Gebhart, G. F., Pain 64, (1996), 493-501) is an animal model of post-operative pain.

In male Sprague Dawley rats (body weight 180-200 g) a longitudinal incision of 1 cm length is made, starting 0.5 cm from the proximal end of the paw, through the skin and fascia. Using bent tweezers the muscle is lifted up from the bottom and incised lengthwise, flat and centered using a scalpel. The muscle is spreaded bluntly with the tweezers. Subsequently, the wound is closed with two sutures.

The tactile hyperalgesia two hours after the operation is determined using an electronic von Frey filament (Somedic Sales AB, Hörby, Sweden). For this purpose the animals are put into a plastic box with a lattice base. With the use of the von Frey filament the paw is subplantar stimulated. In order to investigate the sensitivity to the mechanical stimulus, the paw retraction threshold is determined on the ipsilateral paw as well as in the same position on the untreated contralateral paw and is given in grams of the applied pressure. The retraction threshold per paw is measured four times and the median value is then determined. The retraction threshold of the ipsi- and contralateral paw is measured prior administration (pre-test value) and at several times after administration of a substance, namely (15, 30 and 60 minutes after administration).

The substances are administered intraperitoneally 15 minutes before the measurement in a volume of 5 ml/kg. A control group of animals is treated with vehicle only. The ED₂₅-values (effective dose at 25% of the maximum effect) of the individual components are determined at their maximum effect. For experimental determination of the ED₂₅ values of the combination of both substances, the substances were administered intraperitoneally into the left and right side of the abdomen.

From the median values the Maximum Positive Effect (% MPE) is determined according to the following formula:

% MPE=100−[(measured value after substance administration−pre-test median)/(pre-test value after operation−pre-test median)*100]

Each group of animals (substance and control) consisted of 10 rats. The median±SEM is calculated from the medians of the individual animals. The significance is determined using two-factor-ANOVA for repeated measurements. The significance of the interaction of substance-administration (treatment), time, time * treatment is analyzed and in case of a significant effect of the treatment a Fischer test followed by a Dunnett test is carried out.

The analysis of the results with respect to a supra-additive effect of the inventive combination comprising the components (a) and (b) is carried out via statistical comparison of the theoretical additive ED₂₅-values with the experimentally determined ED₂₅-values of the method according to Tallarida et al. (isobolographic analysis according to Tallarida J T, Porreca F, and Cowan A. Statistical analysis of drug-drug and site-site interactions with isobolograms. Life Sci 1989; 45: 947-961).

The experimental ED₂₅ value was determined with a fixed ration of the components which corresponded to the ratio of the individual ED₂₅ values. The results of the isobolographic analysis are summarized in the following table 1.

TABLE 1
Experimental ED₂₅ values of (1RS,3RS,6RS)-6-Dimethylaminomethyl-1-(3-
methoxy-phenyl)-cyclohexane-1,3-diol hydrochloride (A) and (S)-Pregablin and
isobolographic analysis of the interaction between A and (S)-pregabaline:

| Substance/ | | | | | |
|---|---|---|---|---|---|
| ED₂₅ | | | | | |
| [mg/kg] | | | Theoretical | Experimental | |
| (confidence | | (S)- | ED₂₅ of the | ED₂₅ of | |
| interval) | A | pregabaline | combination | combination | Interaction |
| A + (S)- | 9.15 | 18.8 | 13.91 | 7.95 | supra- |
| pregabaline | (8.09-10.3) | (15.1-22.4) | (12.37-15.46) | (5.65-10.2) | additive |
| | | | | | (p = 0.001) |

p: level of statistical significance

The ratio of A and (S)-pregabaline used in the afore mentioned experiment was 1:2.

### 2. Spinal Nerve Ligation (Chung Model for Mononeuropathic Pain) in Rats

Under pentobarbital anaesthesia (Narcoren®, 60 mg/kg i.p., Merial GmbH, Hallbergmoos, FRG), the L5, L6 spinal nerves were tightly ligated (Kim and Chung, Pain 1992; 50: 355). The left L5 and L6 spinal nerves were exposed by removing a small piece of the paravertebral muscle and a part of the left spinous process of the L5 lumbar vertebra. The L5 and L6 spinal nerves were then carefully isolated and tightly ligated with silk (NC-silk black, USP 5/0, metric 1, Braun Melsungen AG, Melsungen, FRG). After checking hemostasis, the muscle and the adjacent fascia were closed with sutures and the skin was closed with sutures.

### Anti-Allodynic Testing

After operation, animals were allowed to recover for one week. Animals develop tactile allodynia which is stable for at least five weeks. For the assessment of tactile allodynia the rats were placed on a metal mesh covered with a plastic dome and were allowed to habituate until the exploratory behaviour diminished. Threshold for tactile allodynia was measured with an electronic von Frey anesthesiometer (Somedic, Sweden). Animals randomly assigned to groups of 10 for each test dose and vehicle, were tested 0.5 h before administration and on several time points after administration. The median of the withdrawal threshold for each animal at a given time is calculated from five individual stimulations with the electronic von Frey filament. Withdrawal thresholds of the injured paws are expressed as % maximal possible effect (% MPE) comparing predrug threshold of Chung-animals (=0% MPE) and control threshold of sham-animals (=100% MPE). A cut-off is set at 100% MPE. The effect of each compound and vehicle is calculated for each testing time point (e.g. 0.5, 1, 3 h post administration) as interindividual % MPE value (±SEM). Anti-allodynic efficacy is defined as increase of ipsi-lateral withdrawal threshold without effect on contra-lateral withdrawal threshold. Ten animals have been used in each group.

### Data Analysis

Effects of drug combinations are compared to the theoretical sum of the effects of each drug tested alone. Drug combination effects which are clearly greater than the sum of the single drug effects are considered to be supra-additive.
Compound A: (1RS,3RS,6RS)-6-dimethylaminomethyl-1-(3-methoxy-phenyl)-cyclohexane-1,3-diol hydrochloride (Axomadol hydrochloride)
Compound B: Gabapentin hydrochloride

TABLE 2

| Treatment | | Effect | |
|---|---|---|---|
| | | % MPE | |
| | | (time after | |
| Compound | Compound | administration | |
| Dose mg/kg | Dose mg/kg | of A) | Comment |

Experiment 1
(combination of A and B)

| | | | |
|---|---|---|---|
| A | Vehicle (20 min | 29 ± 6 (30 min) | |
| 4 mg/kg iv | before A) | | |
| Vehicle iv | B | 16 ± 5 (30 min) | |
| | 10 mg/kg iv (20 min | | |
| | before vehicle) | | |
| A | B | 67 ± 7 (30 min) | Supra-additive vs |
| 4 mg/kg iv | 10 mg/kg iv (20 min | | 29 + 16 = 45 |
| | before A) | | |

### 3. Randall-Selitto Test in Rats

The weight ratios of the components (a) and (b) that will lead to a supra-additive effect (synergistic effect) of the inventive pharmaceutical composition may be determined via the test of Randall and Selitto as described in Arch. Int. Pharmacodyn., 1957, 111: 409 to 419, which is a model for inflammatory pain. The respective part of the literature is hereby incorporated by reference and forms part of the present disclosure.

Acute inflammation was induced by an intraplantar injection of 0.1 ml of a carrageenan solution (0.5% in distilled water) into one hind paw. The mechanical nociceptive threshold was measured 4 hours after carrageenan injection using an Algesiometer (Ugo Basile, Italy). The device generates a mechanical force with a linear increase over time. The force is applied to the dorsal surface of the inflamed rat hind paw via a cone-shaped stylus with a rounded tip (2 mm tip diameter). The nociceptive threshold is defined as the force (in grams) at which the rat vocalises (cut-off force 250 g). The mechanical nociceptive threshold is measured at different timepoints after the drug or vehicle administration. The antinociceptive and antihyperalgesic activity of the tested substance is expressed as percentages of the maximal possible effect (% MPE). The group size is n=10.

The analysis of the results with respect to a supra-additive effect of the inventive pharmaceutical composition comprising the components (a) and (b) is carried out via statistical comparison of the theoretical additive ED₅₀-value with the experimentally determined ED₅₀-value of a so-called fixed ratio combination (isobolographic analysis according to Tallarida J T, Porreca F, and Cowan A. Statistical analysis of drug-drug and site-site interactions with isobolograms. Life Sci 1989; 45: 947-961).

The interactions studies presented herein were performed using equieffective doses of the two components, calculated from the ratio of the respective ED₅₀ values of the components if administered alone.

The application route was intravenous (i.v.) for Axomadol hydrochloride (A) and intraperitoneal (i.p.) for the anticonvulsants Lamotrigine, Lacosamide, Levetiracetam, Retigabine and Carbamazepine. When A was applied alone, the peak effect was reached 15 min p. appl. (timepoint of first measurement) and ED₅₀-values of 15.26 (14.18-16.75) and 13.60 (13.02-14.16) mg/kg i.v. were calculated. The anticonvulsants induced dose-dependent analgesic effects with ED₅₀-values of 35.3 (32.7-38.1) mg/kg i.p. (Lamotrigine), 35.85 (34.74-36.89) mg/kg i.p. (Lacosamide), 986.0 (854.0-1137.3) (Levetiracetam) mg/kg i.p. and 5.09 (4.68-5.50) mg/kg i.p. (Retigabine) and 30 (28.7-31.5) mg/kg i.p. (Carbamazepine), reaching the peak effect 15 min p. appl. According to their respective timepoint of peak effect, A was applied 15 min and the anticonvulsant component also 15 min before timepoint of measurement of the interaction-experiments (i.e. both components were applied simultaneously). Thus, the time point of ED₅₀ calculation of the combination corresponds to the timepoint of the peak effect of the respective compound. The isobolographic analysis revealed that the experimental ED₅₀-values of the combinations were significantly lower than the respective theoretical ED₅₀-values. Thus, the combination studies demonstrate supra-additive interaction of A with all of the anticonvulsants, Lamotrigine, Lacosamide, Levetiracetam, Retigabine and Carbamazepine.

The results of the isobolographic analysis are summarized in the following Table 3:

TABLE 3
Experimental ED₅₀ values of A, Lamotrigine, Lacosamide, Levetiracetam,
Retigabine and Carbamazepine and isobolographic analysis of the interaction
between A with these anticonvulsants, respectively:

| Substance/ | | | | | |
|---|---|---|---|---|---|
| ED₅₀ [mg/kg] | A | Lamotrigine | Lacosamide | Levetiracetam | Retigabine |
| A + | 15.3 | 35.3 | — | — | — |
| Lamotrigine | (14.2-16.8)* | (32.7-38.1) | | | |
| A + | 15.3 | — | 35.9 | — | — |
| Lacosamide | (14.2-16.8)* | | (34.7-36.9) | | |
| A + | 15.3 | — | — | 986.0 | — |
| Levetiracetam | (14.2-16.8)* | | | (854.0-1137.3) | |
| A + | 13.6 | — | — | — | 5.09 |
| Retigabine | (13.0-14.2) | | | | (4.68-5.50) |
| A + | 15.3 | — | — | — | — |
| Carbamazepine | (14.2-16.8)* | | | | |

| | | Theoretical | Experimental | |
|---|---|---|---|---|
| Substance/ | | ED₅₀ of the | ED₅₀ of | |
| ED₅₀ [mg/kg] | Carbamazepine | combination | combination | Interaction |
| A + | — | 25.3 | 17.1 | supra-additive |
| Lamotrigine | | (23.8-26.7) | (15.8-18.3) | (p < 0.001) |
| A + | — | 25.6 | 17.7 | supra-additive |
| Lacosamide | | (24.4-26.7) | (16.8-18.6) | (p < 0.001) |
| A + | — | 500.6 | 408.5 | supra-additive |
| Levetiracetam | | (459.5-541.8) | (382.8-431.7) | (p < 0.001) |
| A + | — | 9.34 | 5.78 | supra-additive |
| Retigabine | | (8.92-9.76) | (5.46-6.06) | (p < 0.001) |
| A + | 30 | 22.6 | 14.4 | supra-additive |
| Carbamazepine | (28.7-31.5) | (21.5-23.7) | (13.3-15.2) | (p < 0.001) |

*identical single-substance group with A for these combinations
p: level of statistical significance of supra-additive interaction

The ratios of A and the respective anticonvulsant component used in the afore mentioned experiments were as follows:

| Combination of A with | Ratio |
|---|---|
| Lamotrigine | 1:2.31 |
| Lacosamide | 1:2.35 |
| Levetiracetam | 1:62.62 |
| Retigabine | 1:0.374 |
| Carbamazepine | 1:1.96 |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A pharmaceutical combination comprising:
(a) at least one 6-dimethylaminomethyl-1-(3-methoxy-phenyl)-cyclohexane-1,3-diol of formula (I) 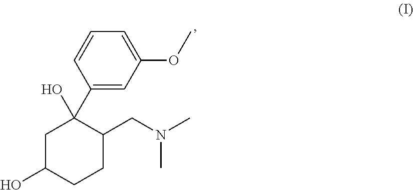
or a salt thereof,
and/or
at least one 6-dimethylaminomethyl-1-(3-hydroxy-phenyl)-cyclohexane-1,3-diol of formula (I′) 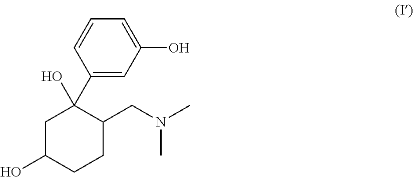
or a salt thereof,
and
(b) at least one antiepileptic.

2. A pharmaceutical combination according to claim 1, wherein component (a) is a mixture of compounds (Ia) and (Ib): 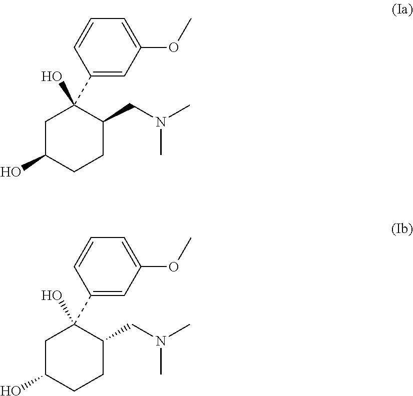
or a salt thereof.

3. A pharmaceutical combination according to claim 2, wherein the mixture of compounds (Ia) and (Ib) is a racemic mixture.

4. A pharmaceutical combination according to claim 1, wherein component (a) is (1RS,3RS,6RS)-6-dimethylaminomethyl-1-(3-methoxy-phenyl)-cyclohexane-1,3-diol hydrochloride.

5. A pharmaceutical combination according to claim 1, wherein the antiepileptic is selected from the group consisting of methylphenobarbital, phenobarbital, primidone, barbexaclone, metharbital, ethotoin, phenyloin, amino(diphenylhydantoin) valeric acid, mephenyloin, fosphenyloin, paramethadione, trimethadione, ethadione, ethosuximide, phensuximide, mesuximide, clonazepam, carbamazepine, oxcarbazepine, eslicarbazepine, rufinamide, valproic acid, valpromide, aminobutyric acid, vigabatrin, progabide, tiagabine, sultiame, phenacemide, lamotrigine, felbamate, topiramate, gabapentin, pheneturide, levetiracetam, brivaracetam, seletracetam, zonisamide, pregabaline, stiripentol, lacosamide, beclamide, retigabine, carisbamate, perampanel and ralfinamide.

6. A pharmaceutical combination according to claim 5, wherein the antiepileptic is selected from the group consisting of pregabaline, gabapentin, topiramate, lamotrigine, carbamazepine, lacosamide, phenyloin, levetiracetam, retigabine, carisbamate, perampanel, valproic acid and ralfinamide.

7. A pharmaceutical combination according to claim 1, wherein components (a) and (b) are present in such a weight ratio that the composition will exert a synergistic effect upon administration to a patient.

8. A medicament comprising a combination according to claim 1.

9. A medicament according to claim 8, wherein said medicament is a solid drug form.

10. A medicament according to claim 8, wherein said medicament is configured for oral administration.

11. A medicament according to claim 8, wherein at least one of the components (a) and (b) is present in controlled-release form.

12. A medicament according to claim 8, wherein components (a) and (b) are each present in controlled-release form.

13. A kit comprising a pharmaceutical combination according to claim 1.

14. A kit according to claim 13, wherein the components (a) and (b) are configured for simultaneous administration.

15. A kit according to claim 13, wherein components (a) and (b) are configured for sequential administration.

16. A kit according to claim 13, wherein the components (a) and (b) are configured for administration by the same pathway of administration.

17. A method of treating or inhibiting pain in a subject in need thereof, said method comprising administering to said subject a pharmaceutically effective amount of a pharmaceutical combination according to claim 1.

18. A method according to claim 17, wherein the components (a) and (b) are administered simultaneously in a combined dosage form.

19. A method according to claim 17, wherein the components (a) and (b) are administered sequentially in separate dosage forms.

20. A method according to claim 17, wherein said pain is selected from the group consisting of inflammatory pain, pain associated with arthrosis, postoperative pain, neuropathic pain, acute pain, chronic pain, visceral pain, headache, migraine pain and cancer pain.
